(19) United States
(12) Patent Application Publication
(10) Pub. No.: US 2008/0020042 A1
(43) Pub. Date: Jan. 24, 2008

(54) Dosage form of sodium ibuprofen

(76) Inventors: Peter Gruber, Merzhausen (DE); Markus Reher, Badenweiler (DE)

Correspondence Address: GIBBONS P.C., ONE GATEWAY CENTER, NEWARK, NJ, 07102, US

(21) Appl. No.: 11/891,430

(22) Filed: Aug. 10, 2007

Related U.S. Application Data
(63) Continuation of application No. 10/683,283, filed on Oct. 14, 2003.

(51) Int. Cl. A61K 9/36; A61K 31/19; A61P 43/00; A61K 9/20

(52) U.S. Cl. 424/479; 424/464; 514/570

(57) ABSTRACT

A non effervescent tablet of ibuprofen, comprising a tablet core and, if desired, a sugar or film coat, wherein the tablet core, based on the weight of the tablet core, consists of 50 to 100% by weight sodium ibuprofen hydrate and 50 to 0% by weight auxiliary material component and contains no lubricant and no disintegrant, and wherein the sodium ibuprofen hydrate has a water content of 8 to 16% by weight, preferably 11 to 16% by weight, possesses a suffcient hardness, is comparably small and leads to a particularly rapid increase in blood level and thereby to an accelerated onset of analgesic effect. Contrary to the current doctrine sodium ibuprofen hydrate having a suitable water content is sufficiently compressible.

The invention relates to a non-effervescent tablet formulation for oral administration of sodium ibuprofen and a process for the production thereof.

Ibuprofen, i.e. 2-(4-isobutylphenyl)propionic acid is a known medicine with analgesic, antiphlogistic and antipyretic properties, that in particular is employed for the treatment of inflammatory diseases and against pain, such as rheumatic diseases, headaches, migraines, toothaches, back aches, muscle pain, post-operative pain and the like. The therapeutically effective form is the S(+)-ibuprofen, whereas the R(−)-enantiomer is practically ineffective, but converts in the body partly into the effective S(+)-form. Although in the last years some preparations have become available in the trade that contain ibuprofen in the S(+)-form, ibuprofen is still employed mostly in racemic form.

An essential point especially in pain treatment is the achievement of a rapid onset of the effect. In order to reach this, the active substance must be rapidly released and absorbed which further requires in the case of solid administration forms that these rapidly disintegrate in the gastrointestinal tract. On the other hand, the solid dosage forms must be small enough that they can still be swallowed without problem.

At the same time, a range of formulation problems arise in the case of ibuprofen. On the one hand, dosage units of ibuprofen formulations typically contain active ingredient quantities corresponding to 200 mg, 400 mg, 600 mg or 800 mg of racemic ibuprofen, i.e. the active ingredient proportion of a tablet must be high, so that it is still swallowable. On the other hand, the formulations must contain sufficient quantities of suitable auxiliary materials, such that the formulations can be compressed in the usual tabletization machinery, do not stick to tabletization tools and result in rapidly disintegrating tablets with sufficient hardness. Moreover, the achievement of a rapid onset of the effect is made more difficult by the fact that ibuprofen is poorly soluble in acidic media, in particular in gastric acid, so that the dissolution and resorption of the active ingredient is considerably delayed.

Active ingredients with a low melting range, such as ibuprofen, can lead to production problems in the tabletization as a consequence of sintering processes and through sticking to the punch and die plates of the tablet press. The sticking can admittedly be rectified by the addition of a large quantity of anti-sticking agents. However in this way the end mixtures become hydrophobic and the release of the active ingredient is slowed thereby. In order to avoid this, and to obtain good flowable powder mixtures that is able to be tabletized, it was proposed in WO-A-93/23026 to mix in dry form 100 parts by weight of ibuprofen or other 2-arylpropionic acids with 50-500 parts by weight of calcium compounds such as calcium hydrogen phosphate, calcium carbonate or calcium hydroxide and to compress this into tablets together with customary auxiliary and/or carrier materials.

In contrast, to improve the ability to be tabletized it was proposed in EP-A1-0 478 838 to convert ibuprofen whole or partly into its calcium salt and to granulate and to tabletize the product by utilizing customary additives and carrier materials, such as microcrystalline cellulose, disintegrants, glidants and lubricants. According to EP-A1-0 478 838 the active ingredient can contain, beside the calcium salt, a portion of ibuprofen or its ammonium, sodium or calcium salt, the ammonium and alkali metal salts, depending on their proportion, improving the solubility but at the same time again increasing the hygroscopicity and the stickiness. The calcium salt that is used to increase the melting range and to improve the ability to be tabletized, is however poorly soluble, and as a result the dissolution and resorption is delayed.

To avoid side effects it was proposed in JP-A-63 198 620 to use ibuprofen together with an antacid (aluminium glycinate, sodium hydrogen carbonate, aluminium lactate and/or a co-precipitate of magnesium hydroxide and potassium sulphate) and/or a coating agent for mucous membranes.

U.S. Pat. No. 4,834,966 described the use of sodium bicarbonate in a water soluble composition, which supposedly gives a drink with a pleasant taste and which contains 33-46% by weight ibuprofen, 34-51% by weight L-arginine and 9-29% by weight sodium bicarbonate.

Furthermore, non-effervescent, water soluble sachet formulations are known from U.S. Pat. No. 5,262,179, which contain a potassium, sodium, arginine, or lysine salt of ibuprofen and a bicarbonate, hydrogen phosphate or tribasic citrate of an alkali metal, in order to mask the taste of the ibuprofen in the aqueous solution. The disclosed formulations are obtained by mere mixing of the components and they contain around 50% by weight or more of further auxiliary materials, in particular dextrose, and only about 20% by weight or less of ibuprofen salt.

In EP-A1-0 607 467, fast releasing S(+)-ibuprofen pellets were described, which contains 0.1-10.0% by weight of basic inorganic salts, such as sodium carbonate, disodium hydrogen phosphate or potassium carbonate, or dilute lye. The latter produces a slight partial dissolving of the S(+)-ibuprofen during the pelleting process, whereupon this becomes slightly sticky, so that the use of the additional binding agent is made unnecessary. The pellets can be provided with customary coatings, in particular protective coatings, gastric juice-resistant coatings or retarding coatings. The coated pellets can, if desired, be compressed by means of conventional processes, to tablets which, per 400 mg S(+)-ibuprofen, contain 73-410 mg, preferably 240-260 mg of tabletting excipients, such as microcrystalline cellulose, starch, croscarmellose sodium, magnesium stearate etc. The fast release of the active ingredient according to USP XXII in phosphate buffer (pH 7.2), as indicated in EP-A1-0 607 467, does however not say anything about the rapidity of the resorption of such a medicinal form under in vivo conditions, as the solubility of ibuprofen and its enantiomers is extremely pH-dependant. Whereas ibuprofen goes quickly into solution by salt formation at pH 7.2, it is only slightly soluble in acidic medium. However, acid conditions dominate in the stomach, and even in the upper intestinal tract, pH values of 7 are in general not reached. This leads to the situation that ibuprofen only gradually goes into solution in the lower intestine through salt formation and therefore a rapid appearance of an active ingredient level is not possible.

Furthermore, a non-effervescent tablet is known from WO-A-97/30699, that contains a ibuprofen medicament in a quantity of at least 35% percent by weight, a carrier material, comprising a compressible filler component in combination with a disintegrant component, and additionally an alkali metal carbonate or bicarbonate in the carrier material in sufficient quantity, that the administration form has a hardness in the range of 6.5-15 kp and a disintegration time of less than 10 minutes, with the proviso that the ibuprofen medicament does not contain a calcium salt of ibuprofen in combination with a alkali metal salt of ibuprofen. According to the disclosure of WO-A-97/30699, the alkali metal carbonates and bicarbonates, which are normally not used as compressible materials, supposedly are suitable to increase the compressibility of compositions that contain a compressible filler in combination with a disintegrant.

The ibuprofen medicament in the dosage form according to WO-A-97/30699 can be ibuprofen, one of its enantiomers or a salt or hydrate thereof. The dosage form is supposedly particularly advantageous to the formulation with the poorly compressible alkali metal salts and especially the sodium salt, that is described as fluffy, soft, sticky, especially poorly compressible and also as having a poor ability to be granulated. As filler, preferably a cellulose derivative, in particular microcrystalline cellulose, and as disintegrant, preferably croscarmellose sodium or sodium starch glycolate, is used. The described formulation can contain further auxiliary materials, such as dilution agents, lubricating agents and flow agents and can have a sugar or film coat. The disclosed formulation examples mostly contain about 50% by weight of sodium ibuprofen dihydrate and about 50% by weight of auxiliary materials, namely microcrystalline cellulose and optionally lactose as fillers, crosslinked polyvinylpyrrolidone or croscarmellose sodium as disintegrant, magnesium stearate, stearic acid or vegetable oil as lubricating agent, alkali metal carbonate or bicarbonate and optionally talc or silicon dioxide as flow agent.

The ibuprofen preparations available on the market (e.g. NUROFEN, Boots) contain the active ingredient mostly in the form of the acid, which however is poorly soluble in acidic media and therefore in the stomach and in the upper intestinal regions. Many attempts have been made to accelerate the resorption and thereby the achievement of a sufficient blood level, in order to obtain a rapid onset of the pain relieving effect. These developments have lead to a range of tablet formulations on the market, that contain, instead of the ibuprofen which is difficult to dissolve in the pH range of stomach acid, ibuprofen lysinate (e.g. DOLORMIN, Woelm Pharma GmbH&Co., Bad Honnef, Germany) or ibuprofen arginate (e.g. DOLO-SPEDIFEN 200, Inpharzam AG, Cadempino, Switzerland). However, the amino acids lysine and arginine are very expensive and increase the price of the corresponding formulations. Moreover, the use of these salts necessitates significantly higher active ingredient quantities and therefore increases the tablet weight. For example, for the 200 mg dosage unit of ibuprofen, the equivalent quantity in the case of the ibuprofen lysinate is 342 mg, and for ibuprofen arginate it is 369 mg. For example, the DOLORMIN tablets corresponding to the 200 mg and 400 mg dosage units of ibuprofen have a tablet weight of 400 mg and 800 mg respectively; in the case of the 400 mg dose it is an oblong tablet with the already considerable dimensions of a length of 19.3 mm, a width of 8.6 mm and a height of 6.6 mm, which can no longer be swallowed by many patients without problem. The DOLO-SPEDIFEN 200 tablet, which corresponds to a 200 mg dosage unit of ibuprofen, has a tablet weight of 610 mg, and thus a corresponding tablet for the double dose is no longer practicable. The use of ibuprofen lysinate or ibuprofen arginate is therefore only for lower dosages a practical, although expensive alternative to the use of ibuprofen.

Also obtainable on the market are soft gelatin capsules (SPALT LIQUA, Whitehall-Much, Münster, Germany) that contain 200 mg dissolved ibuprofen and a small amount of a potash lye for the purpose of rapid resorption. However an expensive, specialised equipment is necessary for the production of the capsules, which only a few specialised firms have available. Moreover, due to the lack divisibility of the capsule, the dosage cannot be individually adapted. Corresponding capsules with 400 mg ibuprofen have hitherto not been available and furthermore would be very big and not very swallow-friendly.

The ammonium and alkali metal salts of ibuprofen are known as sticky, hygroscopic and poorly compressible substances. In particular, the sodium salt, due to its waxy nature, is regarded as exceptionally poorly compressible and also as having a poor ability to be granulated (K. D. Rainsford, “Ibuprofen: A critical bibliographic review”, Publisher: Taylor & Francis, 1999, ISBN 0-7484-0694-8, page 75). This is also the reason that hitherto no sodium ibuprofen containing tablets have been available on the market.

The object of this invention is to provide a technically feasible manufacturable tablet formulation, that permits a rapid release and resorption of the active ingredient and that nevertheless allows comparatively small tablet sizes.

The object is achieved through a non-effervescent tablet for oral administration of sodium ibuprofen, comprising a tablet core and, if desired, a sugar or film coating on the tablet core, wherein the tablet core consists of, based on the weight of the tablet core, from 50 to 100% by weight sodium ibuprofen hydrate and 50 to 0% by weight auxiliary material component and contains no lubricant and no disintegrant, the sodium ibuprofen hydrate having a water content from 8 to 16% by weight of the hydrate.

Surprisingly it was found that the ability of sodium ibuprofen to be tabletized heavily depends on its water content and, contrary to current opinion, it is possible to produce tablets with sufficient hardness and short disintegration times, that contain comparatively little or no auxiliary material, if a sodium ibuprofen hydrate is used with a water content of 8 to 16% by weight, preferably 11 to 16% by weight and the water content is precisely controlled. Due to the particularly poor compression properties that were described in the state of the art and the waxy nature, a person skilled in the art would normally never try to produce a tablet which is largely free of auxiliary material, but would add comparatively high quantities of compressible fillers and disintegrants, in order to obtain, nevertheless, useful compression and disintegration properties. It was therefore completely unexpected, that by means of suitable water content, even tablets out of pure sodium ibuprofen hydrate can be produced.

### IN GRAPHICAL FORM

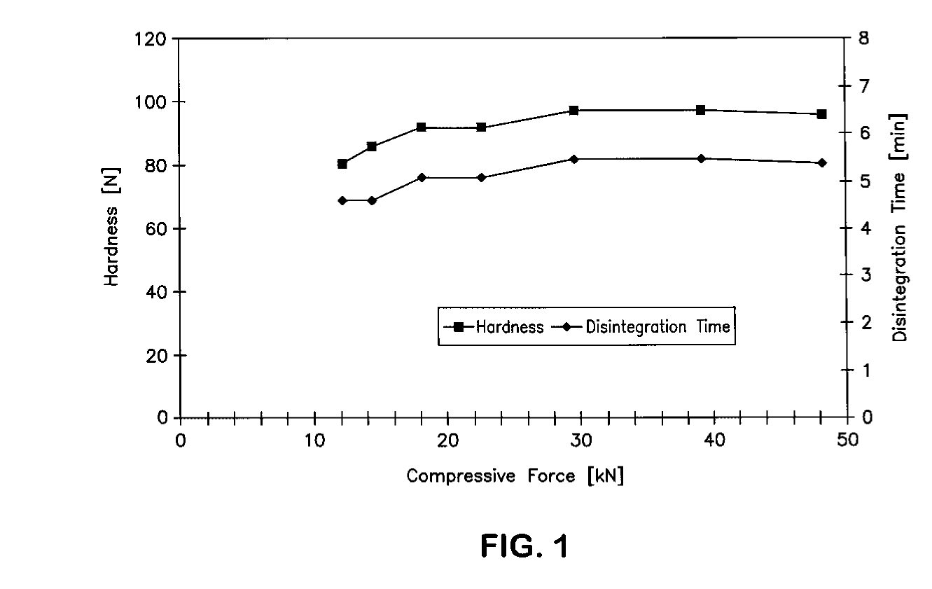

FIG. 1 shows the hardness and the disintegration time of a tablet of this invention in relation to the compressive force used in the tabletization process,

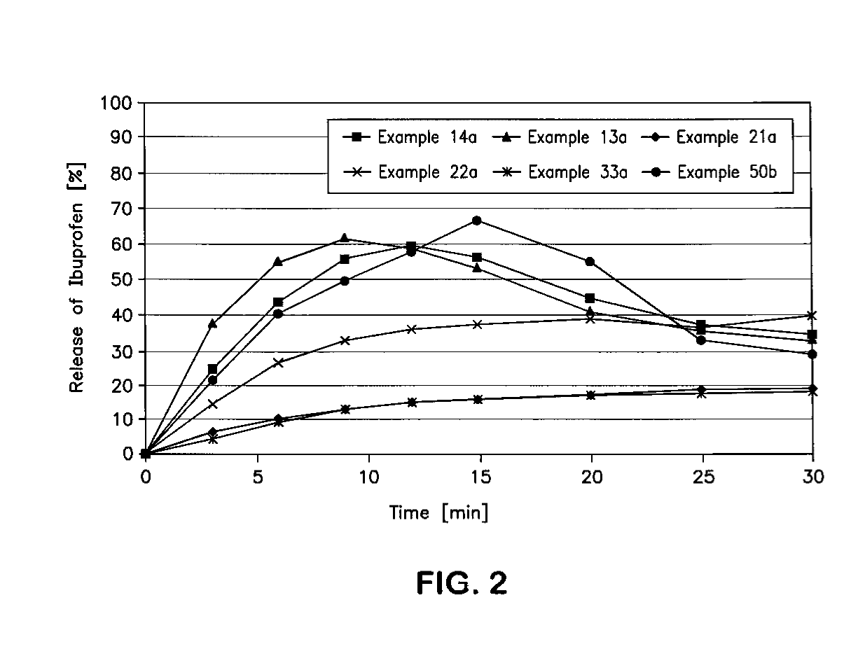

FIG. 2 shows the dissolution profile of tablets of this invention in 0.1 M hydrochloric acid (pH 1.2) according to the Paddle-Method at 50 rpm,

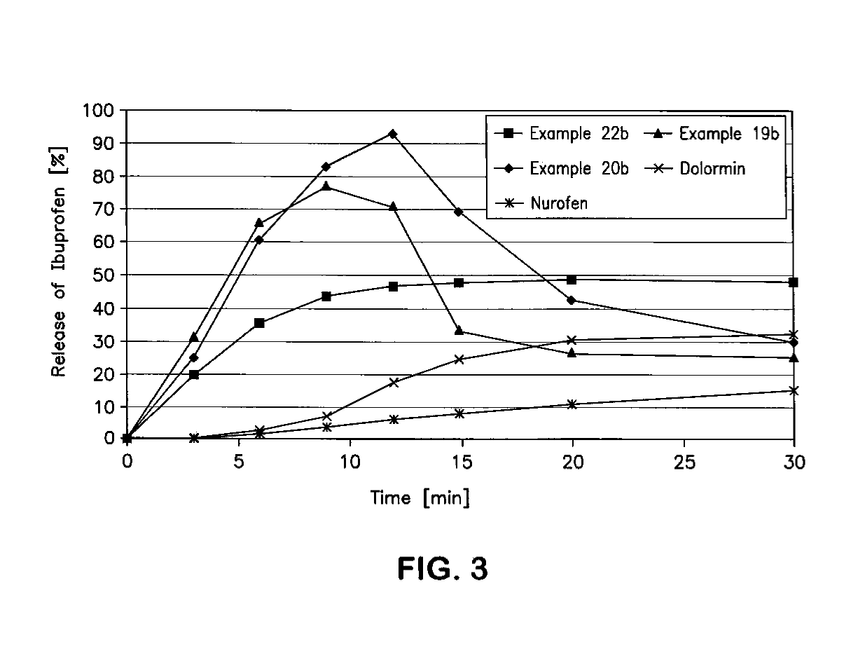

FIG. 3 shows the dissolution profile of film tablets of this invention in 0.1 M hydrochloric acid (pH 1.2) according to the Paddle Method at 100 rpm in comparison to a Dolormin film tablet and a Nurofen film tablet,

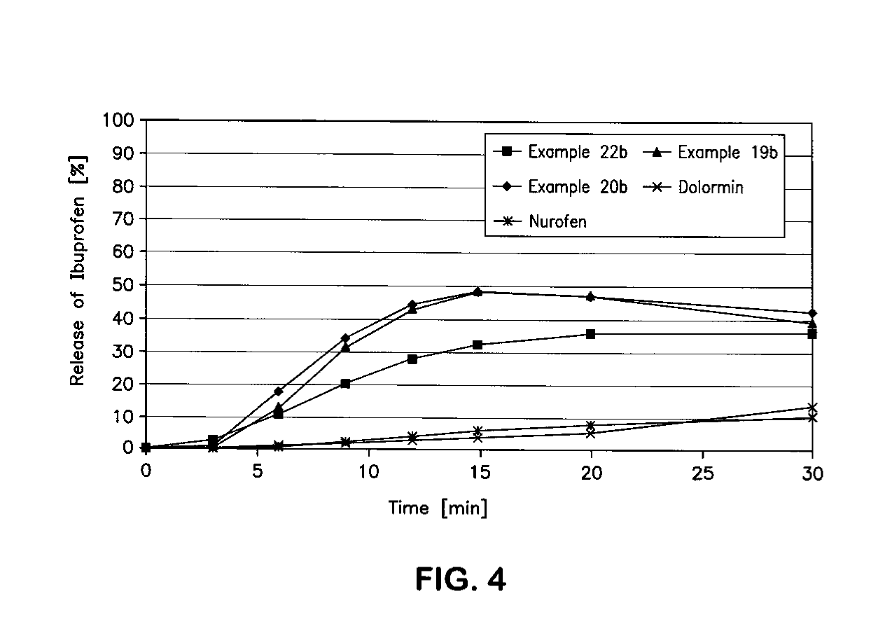

FIG. 4 shows the dissolution profile of film tablets of this invention in McIlvain buffer (pH 3.5) according to the Paddle Method at 100 rpm in comparison to a Dolormin film tablet and a Nurofen film tablet, and

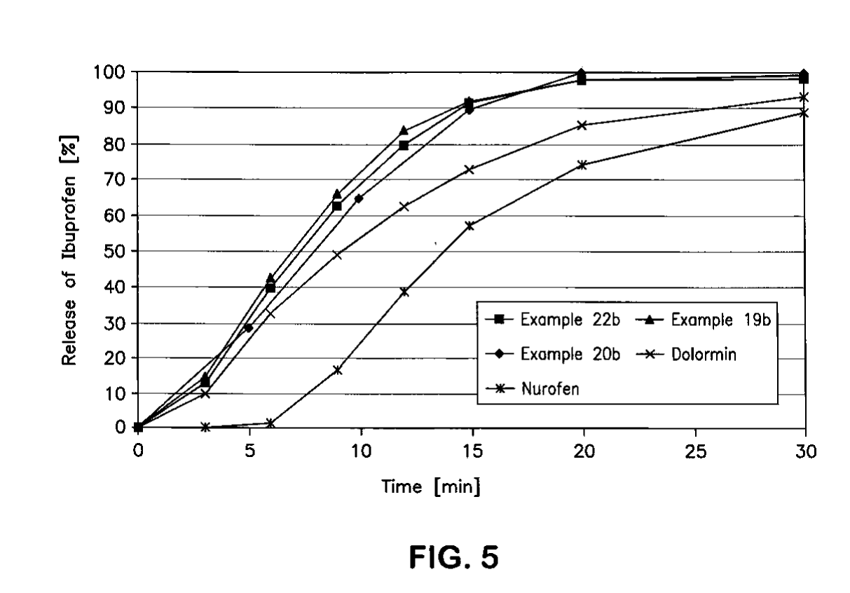

FIG. 5 shows the dissolution profile of film tablets of this invention in USP buffer (pH 7.2) according to the Paddle Method at 50 rpm in comparison to a Dolormin film tablet and a Nurofen film tablet.

In principle sodium ibuprofen can be water free, or can exist as the mono- or dihydrate, or as a mixture of these forms. The water free form and the monohydrate are hygroscopic and take up water, resulting in the formation of the dihydrate. For example, water free sodium ibuprofen spontaneously takes up to about 13.6% by weight of water already at a relative humidity level of 25% RH. Therefore, if the monohydrate were used, a hygroscopic tablet would result and a very dense packing material would be necessary; otherwise the tablet would strongly absorb water, soften and have a tendency to capping. Moreover, in the case of the film tablets, the expansion of the tablet due to the uptake of water would be so great that the film coat would burst open.

On the other hand, the dihydrate is practically no longer hygroscopic and absorbs less than 0.5% by weight of additional water, at room temperature with a relative humidity level of 90% RH. For example, sodium ibuprofen hydrate with a water content in the range of 13-14% by weight, did not take up additional water during open storage for over 6 months at 40° C. and 75% RH. It is quite common, that the sodium ibuprofen dehydrate is delivered from the manufacturer with a water content not corresponding to the dihydrate, as the water of crystallization is easily lost upon drying at 40-50° C. and the substance easily changes into the monohydrate form by drying. This fact illustrates the importance of a precise control of the water content, and it may also explain why the dependence of the ability to be tabletized on the water content has not been discovered in the art.

If the water content of the sodium ibuprofen hydrate is less than 11% by weight, it is increasingly difficult to produce sufficiently hard tablets which do not have a tendency to capping and to avoid the sticking on the tabletization tools. If the water content is about 8 to 11% by weight, these disadvantages can be compensated to a large extent through the addition of suitable auxiliary materials. On the other hand, if the water content of the sodium ibuprofen hydrate is about 5% by weight or less, it is practically no longer possible to produce a tablet with little auxiliary material.

Surprisingly, it was furthermore found that the hardness and disintegration time of the tablets of this invention are nearly independent of the compressive force used during tabletization despite the lack of a disintegrant. FIG. 1 illustrates in graphical form the hardness measured by means of a Schleuniger Hardness Tester and the disintegration time measured in water by 37° C. in relation to the compressive force used for a tablet of this invention, consisting of 512.5 mg sodium ibuprofen hydrate (water content between 13 and 14% by weight), 50 mg polyvinylpyrrolidone K25 and 99.5 mg sodium hydrogen carbonate. As is apparent, an increase of the compressive force used from 20 to 50 kN only leads to an insignificant increase of the hardness and the disintegration time. Owing to this unexpected finding, it can be practically ruled out that tablets which are too hard and with impaired disintegration and release properties will result from the use of too much pressure, which additionally facilitates the production of the tablets of this invention.

Furthermore, it was unexpectedly found that the tablets of this invention can be produced without the addition of an inner lubricant such as magnesium stearate, calcium stearate, stearic acid, fat triglycerides and the like. As is known, lubricants must usually be added to the tablet mixtures, so that there is no sticking to the tabletization tools and the friction is not too great when the tablet is ejected. Without the use of a lubricant, considerable disturbance to the tabletization process normally results, which has the consequence that the tablet press must be turned off and the tablets are unusable, as they are injured by the ejection from the machinery. It was therefore completely surprising that lubricants could be dispensed with in the production of tablets of this invention and that by using customary tablet presses, millions of tablets could be pressed without any addition of an inner lubricant. In fact it was found that addition of classic lubricants such as magnesium stearate even increases the danger that the final mixture sticks to the surface of the punch. Moreover, the customary lubricants are hydrophobic and would decrease the compressibility and the disintegration properties. Therefore, the tablet formulations of this invention expediently do not contain significant quantities (i.e. less than 0.1% by weight) of lubricant in the tablet core, and they are advantageously completely free of inner lubricants.

Further it turned out as a consequence of the absence of inner lubricants, that it is also no longer required to add a disintegrant to the tablet mix. The proportion of auxiliary materials can thereby be further reduced or even completely eliminated. The water solubility of the sodium ibuprofen hydrate is actually so great, that the disintegration of the tablet cannot be improved through the addition of customary disintegrants or combinations of fillers such as microcrystalline cellulose with disintegrants. Therefore, the tablet formulations of this invention expediently do not contain significant quantities (i.e. less than 0.1% by weight) of disintegrants or fillers with disintegrant properties, such as crosslinked polyvinylpyrrolidones, magnesium aluminium silicates, microcrystalline cellulose, starches, sodium carboxymethylcellulose starches etc., and advantageously they are completely free of such materials.

The disintegration times of the tablets of this invention are generally significantly below 10 minutes, typically in the range from about 2 to 7 minutes. Owing to the high water solubility of the sodium ibuprofen hydrate and the elimination of an inner lubricant, the tablets of this invention enable a particularly rapid release and resorption of the active ingredient, which leads to a rapid increase of the blood level and concentration at the site of effect. Furthermore, it was found that the tablets of this invention, particularly if they contain a basic component, can lead to significantly supersaturated solutions in acidic medium, which additionally aids a rapid resorption. In comparison to known ibuprofen medicines, the present invention therefore achieves more rapidly effective blood levels and concentrations at the site of effect, and thereby an accelerated onset of the analgesic effect, as well as a rapider achievement of the maximal blood levels and concentrations at the site of effect. Through numerous in vivo studies it has been verified that the maximal blood level is achieved with conventional ibuprofen formulations only about 1.5 hours after administration. In contrast, maximal blood levels were already achieved after about 35 minutes with the tablets of this invention without disintegrant. The tablets of this invention therefore permit an especially rapid treatment of pains and lessen the danger that the patient takes another tablet as a result of a too slow onset of the analgesic effect.

The elimination of lubricant and disintegrant and the reduction or elimination of further auxiliary materials in the tablet formulation of this invention enables a significant decrease of the tablet weight and size. Since the quantity of sodium ibuprofen dihydrate equivalent to 200 mg ibuprofen is only 256 mg, the weight difference to the insoluble ibuprofen is not too great, whereas in comparison to the soluble ibuprofen lysinate and the soluble ibuprofen arginate a clear weight reduction is achieved also in respect of the active ingredients. Consequently, the tablets of this invention are rapidly resorbed, as well as being comparatively small.

Since the production of the tablets can be carried out in a manner known per se with conventional tablet presses, the proportion of auxiliary materials can be kept low, and the active ingredient costs are low in comparison with the lysinate and arginate, the production of tablets of this invention is particularly economically feasible.

The expression “tablet core” indicates in the context of the present invention a tablet without sugar or film coat.

The expression “sodium ibuprofen hydrate” in the context of the present invention comprises the sodium salt of racemic ibuprofen, as well as the sodium salts of the enantiomers S(+)-ibuprofen and R(−)-ibuprofen and of mixtures of these enantiomers. Preferably used are S(+)-sodium ibuprofen hydrate and, in particular, racemic sodium ibuprofen hydrate. The water content of the hydrate is expediently about 8 to 16% by weight, preferably about 11 to 16% by weight, based on the weight of the hydrate; particularly preferred is a water content of about 12.5 to 15% by weight, more particularly about 13 to 14% by weight. Owing to the water content in accordance with this invention, the hydrate exists predominately or entirely in the dihydrate form. Whereas lower proportions of monohydrate hardly proved to have a disturbing effect, the ability to be tabletized is reduced with increasing monohydrate proportions, which must be compensated to a certain degree by auxiliary materials.

In the context of the present invention the water content of the sodium ibuprofen hydrate was determined in each case as loss on drying at 105° C., since the water of crystallisation is completely lost at this temperature.

The proportion of sodium ibuprofen hydrate in the table: formulations of this invention, is expediently about 50 to 100% by weight, preferably about 60 to 100% by weight and especially preferably about 70 to 100% by weight, based on the weight of the tablet core. Correspondingly the proportion of auxiliary material in the tablet core is expediently about 50 to 0% by weight, preferably about 40 to 0% by weight and especially preferably about 30 to 0% by weight.

According to a preferred embodiment, the tablet core can essentially consist of sodium ibuprofen hydrate and be essentially free of auxiliary materials, i.e. it can contain preferably less than 0.1% by weight or especially preferably no auxiliary materials. In this embodiment, the water content of the sodium ibuprofen hydrate should be preferably about 11 to 16% by weight, a water content of about 12.5 to 15% by weight, in particular about 13 to 14% by weight, being especially preferred. Furthermore, the tablets should have a tablet hardness (measured by means of a Schleuniger Hardness Tester) of preferably at least about 30 N, especially preferably at least about 40 N.

However, in general it is preferred to employ in the tablet core a small proportion of at least about 0.1% by weight of auxiliary material, which expediently exists in mixture with the sodium ibuprofen hydrate. Therefore, in the case of the tablet cores which contains auxiliary material, the proportion of sodium ibuprofen hydrate can desirably be about 50 to 99.9% by weight, preferably about 60 to 99.9% by weight and especially preferably about 70 to 99.9% by weight, based on the weight of the tablet core. Correspondingly, the proportion of auxiliary material in the tablet core desirably amounts to about 50 to 0.1% by weight, preferably about 40 to 0.1% by weight and especially preferably about 30 to 0.1% by weight.

In principle, the auxiliary material that can be used in the tablet core can be water soluble or poorly water soluble or insoluble materials. For example, it can occasionally be desirable to use in the tablet mix an insoluble binding agent such as silicon dioxide. In general, it is however preferred to use predominately or exclusively water soluble auxiliary materials in the tablet core. In the context of the present invention, “water soluble” describes those materials that are soluble in water at 25° C. in a concentration of at least about 1% by weight.

The proportion of auxiliary materials (which preferably can be water soluble), can preferably be about 7 to 40% by weight, especially preferably about 15 to 30% by weight, and in particular about 20 to 25% by weight, based on the weight of the tablet core. Therefore the active ingredient proportion in the tablet core can preferably amount to about 60 to 93% by weight, especially preferably about 70 to 85% by weight, and in particular about 75 to 80% by weight.

Preferably suitable as the auxiliary material component in the tablet core are fillers and/or basic auxiliary materials. Furthermore, if desired, the tablet core can contain a low quantity of surfactant.

Preferably suitable basic auxiliary materials are such materials which give, in a concentration of 1% by weight in water at 25° C., an aqueous solution or suspension with a pH value of at least 7.5. Examples of preferably suitable basic auxiliary materials are basic alkali metal salts, basic alkaline earth metal salts and basic ammonium salts, for example in the form of the carbonates, hydrogen carbonates, phosphates, hydrogen phosphates, oxides, hydroxides, citrates, tartrates, acetates or propionates, in particular basic sodium salts, basic potassium salts and basic ammonium salts, such as sodium hydrogen carbonate, potassium hydrogen carbonate, sodium carbonate, potassium carbonate, ammonium carbonate, trisodium citrate, disodium tartrate, dipotassium tartrate, magnesium oxide, calcium oxide, magnesium hydroxide, calcium hydroxide, magnesium carbonate, calcium carbonate, disodium hydrogen phosphate, dipotassium hydrogen phosphate, trisodium phosphate, tripotassium phosphate, tricalcium phosphate, sodium acetate, potassium acetate, sodium propionate etc., basic amino acids, such as lysine and arginine, and the like. In general, the water soluble, basic auxiliary materials such as sodium hydrogen carbonate, potassium hydrogen carbonate, sodium carbonate, potassium carbonate, trisodium citrate and trisodium phosphate are preferred. Especially preferably used are sodium hydrogen carbonate, potassium hydrogen carbonate or a mixture of both, in particular sodium hydrogen carbonate.

The basic auxiliary materials aid the formation of a weekly basic micro milieu on the tablet surface and thereby presumably counteract a rapid precipitation of the ibuprofen in the stomach. The proportion of the basic auxiliary material in the tablet core may, if present, preferably be about 5 to 30% by weight, in particular about 6 to 25% by weight, based on the weight of the tablet core. Typically, about 8 to 20% by weight of basic auxiliary material is mostly used, in particular about 13 to 17% by weight. If the basic auxiliary material is a hydrogen carbonate, such as sodium or potassium hydrogen carbonate, the proportion may preferably amount to less than 1 molar equivalent, for example about 0.2 to 0.8 molar equivalent, in regard to the sodium ibuprofen hydrate.

As filler in the tablet core generally auxiliary materials that improve the compressibility are suitable. However preferably in general are neutral to weakly acidic fillers that improve the compressibility, preferably those that do not have a buffering effect. In the context of the present invention the expression “neutral to weakly acidic filler” comprises in particular fillers that, at a concentration of 1% by weight in water at 25° C., result in an aqueous solution or suspension with a pH value between 4 and 7.5. Preferably water soluble fillers are used. Examples of preferably suitable fillers are sugars such as saccharose, glucose, fructose and lactose, hexoses such as mannitol, xylitol, maltitol, sorbitol, hydrolysed or enzymatically split starch such as maltodextrin, cyclodextrins such as β- and γ-cyclodextrin, non-crosslinked (water soluble) polyvinylpyrrolidone, polyvinyl alcohols, polyethylene glycols, polypropylene glycols, alkali metal salts, alkaline earth metal salts and ammonium salts of organic or inorganic acids, in particular sodium, potassium, magnesium and calcium salts such as sodium chloride, potassium chloride, magnesium chloride, sodium sulphate, potassium sulphate, magnesium sulphate, trimagnesium dicitrate, tricalcium dicitrate, calcium lactate, calcium gluconate, calcium hydrogen phosphate and the like. Especially preferred fillers are hexoses such as sorbitol and mannitol, non-crosslinked polyvinylpyrrolidone, maltodextrin and sodium chloride, in particular water soluble, non-crosslinked polyvinylpyrrolidone, which is apparently also suitable to delay the precipitation of the ibuprofen in the stomach. Povidones K25-K90 (BASF, Germany) such as Povidone K25 and Povidones K29-32 are, for example, suitable as water soluble, non-crosslinked polyvinylpyrrolidones.

The proportion of the filler in the tablet core can, if present, preferably amount to about 1 to 25% by weight, in particular about 3 to 20% by weight and typically about 5 to 15% by weight, based on the weight of the tablet core.

The tablet formulation of this invention can contain fillers or basic auxiliary materials or both. If the tablet core contains filler as well as basic auxiliary material, the optimal quantity can occasionally be a little lower than the aforementioned quantities. Furthermore the total quantity of filler and basic auxiliary materials expediently amounts to at the most about 50% by weight, preferably at most about 40% by weight and especially preferably at most about 30% by weight, based on the weight of the tablet core.

According to a particularly preferred embodiment, the tablet formulation of this invention contains as the auxiliary material component sodium hydrogen carbonate and/or potassium hydrogen carbonate and non-crosslinked polyvinyloyrrolidone. Preferably the formulation can contain, based on the weight of the tablet core, about 5 to 15% by weight, in particular about 5 to 10% by weight, of non-crosslinked polyvinylpyrrolidone and about 7 to 20% by weight, in particular about 12 to 18% by weight of sodium hydrogen carbonate and/or potassium hydrogen carbonate. Preferably the tablet core contains no further auxiliary materials, i.e. the tablet core can preferably consist of sodium ibuprofen hydrate, non-crosslinked polyvinylpyrrolidone, and sodium hydrogen carbonate and/or potassium hydrogen carbonate.

If desired, the tablet mixture can also contain a surfactant such as sodium dodecylsulfate as auxiliary material. However, the proportion of surfactant, if present, is in general not over about 2% by weight and can typically amount to about 0.1 to 2% by weight, for example about 1% by weight, based on the weight of the tablet core. The addition of a surfactant is however generally not required, which is why the tablet core of this invention can preferably be surfactant free. Therefore the auxiliary material component can preferably consist of basic auxiliary material and/or neutral to weakly acidic filler that improves the compressibility, i.e. the tablet core preferably consists of the sodium ibuprofen hydrate and basic auxiliary material and/or neutral to weakly acidic filler that improves the compressibility.

If the water content of the sodium ibuprofen hydrate is under 11% by weight, i.e. in the range between 8 and 11% by weight, a comparatively high auxiliary material proportion is in general indicated, in order to counteract the reduction of the properties of the sodium ibuprofen hydrate. Therefore in this case in general a proportion of auxiliary material, in particular filler and/or basic auxiliary material, of about 30 to 50% by weight, based on the weight of the tablet core, is preferred.

The tablets of this invention can contain the active ingredient sodium ibuprofen hydrate in conventional dosages, high doses also being possible due to the low proportion of auxiliary material. Therefore the tablets of this invention can contain for example about 128 mg to 1024 mg of sodium ibuprofen hydrate (corresponding to 100 mg to 801 mg ibuprofen), in which dosages in the range of about 256 mg to 768 mg, in particular about 256 mg to 512 mg, are in general preferred.

The tablet formulations of this invention can preferably be coated with a sugar or film coating, in which all customary sugar and film coating materials are in principle suitable as coating materials. The thickness of the coat is not critical; however in general the proportion of the coat, based on the weight of the tablet core, is only about 1 to 10% by weight, preferably about 3 to 6% by weight.

The tablets of this invention can be produced by compressing the sodium ibuprofen hydrate, optionally in mixture with auxiliary material, into tablet cores and, if desired, coating the tablet cores with a sugar or film coating. The tabletization can be carried out in a manner known per se with customary tablet presses. Likewise, a sugar or film coat can be applied in a manner known per se by conventional methods. Attention should however be paid during production to ensure that the water content of the sodium ibuprofen hydrate lies in the aforementioned ranges.

In general it is preferred that, prior to tabletization, sodium ibuprofen hydrate is granulated in dry form, optionally together with the auxiliary material or a part of the auxiliary material. If the sodium ibuprofen hydrate shows a bulk volume of more than 0.35 ml/g the granulation can, if desired, be dispensed with. To determine the bulk volume, a 250 ml measuring cylinder is carefully and slowly filled up, without shaking, with an exactly weighed quantity of substance. Lastly, the poured in substance is levelled off, if necessary by using a hairbrush to level off the surface of the substance in the cylinder, and the volume of the substance is read off. The bulk volume is the quotient of the read off volume and the mass of the introduced substance.

If auxiliary materials, in particular filler and/or basic auxiliary material are used, these can be admixed before the granulation, or just be admixed to the final mixture directly prior to tabletization, or a part of the auxiliary materials can be employed in the granulation and the rest added to the final mixture. However, if the tablet contains filler as well as basic auxiliary material, in general it is preferred, to add the filler already in the granulation and the basic auxiliary material only in the final mixture.

The invention also concerns a method to achieve an accelerated onset of analgesic effect, comprising the production of the tablets of this invention and the administration thereof to a patient suffering from pain.

The invention is further illustrated by the following examples. In the examples, Kollidon CL (Hoescht, Germany) denotes a water insoluble, crosslinked polyvinylpyrrolidone; Povidone K25-K90 (BASF, Germany) denotes water soluble, non-crosslinked polyvinylpyrrolidones; dimethicone (Wacker, Germany) is a silicone oil; Hypromellose 2910, 6 and 15 mPas (Shin Etsu, Japan) is a water soluble hydroxypropylmethylcellulose; Magrogol 4000 and Magrogol 6000 (Hoechst, Germany) is a highly polymerised, waxy and water soluble polyethylene glycol with an average molecular weight of 4000 to 6000 respectively; and titane dioxide (Schweizerhalle, Switzerland) is a water insoluble white pigment.

### EXAMPLE 1

a) 256.25 kg sodium ibuprofen dihydrate were mixed homogenously in a conventional mixer with 25.0 kg Povidone K25 for 10 minutes. This mixture was compacted in a roller compactor, and the compacted material was broken over a sieve with the mesh width of 1.0 mm. Portions with a granular size under 0.25 mm were once more compacted and broken.

49.75 kg sodium hydrogen carbonate, sieved through a sieve with mesh width of 0.71 mm, were mixed in a conventional mixer with the compacted material for 10 minutes. The obtained final mixture was compressed on a rotary press with 16 presses at an average hourly output of 50 000 tablets. The obtained oval, biconvex tablets had a weight of 331 mg, a length of 11.7 mm a width of 7.7 mm and a height of 4.6 mm.

To determine the hardness of the tablets, the necessary force to crush the tablet between the motorised jaws of a Schleuniger Hardness Tester was measured. The average hardness (mean from 10 measurements) was 78 N.

The disintegration time of the tablets was measured by means of the disintegration method described in the European Pharmacopoeia, 4^{th} edition, Chapter 2.9.1, page 191, using water (pH about 7) as disintegration medium. The average disintegration time of the tablets (mean from 6 measurements) was 5.2 minutes.

b) 331 kg of the obtained tablets were loaded in a Glatt Coater and sprayed with a solution of 3.5 kg Hypromellose 2910, 0.75 kg lactose monohydrate and 0.75 kg Magrogol 6000 in 10 kg water and 40 kg ethanol (96%) at a product temperature of 35° C. to 42° C., and isolated. Under the same conditions, the isolated film tablet cores were sprayed with a suspension of 2.8 kg Hypromellose 2910, 3.6 kg lactose monohydrate, 1.0 kg Magrogol 4000 and 2.6 kg titane dioxide in 56 kg water and 24 kg ethanol (96%). The dried film tablets were treated with a polishing solution of 2 kg Magrogol 6000 and 17 kg water. The final weight of the film tablets was 348 mg.

### EXAMPLE 2

As described in Example 1, 331 kg of the final mixture for tabletization was produced. In an analogous manner to Example 1, this was compressed to form oblong, biconvex tablets with break score on one side, and the tablets obtained were processed to film tablets as described in Example 1. The tablet cores had a weight of 662 mg, a length of 17.3 mm, a width of 8.3 mm a height of 5.0 mm and a content of sodium ibuprofen dihydrate of 513 mg (corresponding to 400 mg ibuprofen acid); the average hardness was 98 N and the average disintegration time was 5.7 minutes. The final weight of the film tablets was 696 mg.

### EXAMPLES 3-50

a) The tablet formulations listed in Table 1 were produced in an analogous manner to Example 1a.

To produce the granulate, the sodium ibuprofen hydrate was mixed with the excipients used in dry granulation (auxiliary materials A), if any, in a conventional mixer for 10 minutes, the obtained mixture or, as the case may be, the sodium ibuprofen hydrate used without auxiliary materials was compacted on a roller compactor, the compacted material was broken over a sieve with the mesh width of 1.0 mm, and portions with a granular size under 0.25 mm were once more compacted and broken. In Example 41, a sodium ibuprofen hydrate with a mean particle size of 0.1-0.2 mm and a bulk volume of over 0.35 g/ml was used and the obtained sodium ibuprofen hydrate/maltodextrin mixture was not compacted, but directly used for tabletting. In the Examples 28-30, a granulate with a granular size of 0.25-1.25 mm (Example 28), 0-0.25 mm (Example 29) or 0-1.25 mm (Example 30) was produced and used in tabletization. The water content of the sodium ibuprofen hydrate used was determined in each case as loss on drying at drying at 105° C.

The obtained granulate (granular size in the range of 0.25 to 1.0 mm, if not otherwise indicated) was mixed in a conventional mixer with auxiliary materials (auxiliary materials B), if any, for 10 minutes. The obtained final mixture (or the granulate itself, if no auxiliary material B was used) was compressed on a rotary press with 16 presses at an average hourly output of 40 000-60 000 tablets. The obtained oval, biconvex tablets had a weight of 300-350 mg, a length of 11.7 mm, a width of 7.7 mm and a height of about 4.6 mm with the press machinery that was used.

The water content of the used sodium ibuprofen hydrate, the proportion of the sodium ibuprofen hydrate in the tablet formulation, as well as the used auxiliary materials A and B and their proportions in the tablet formulation are compiled in Table 1.

TABLE 1

| | | % weight of | % weight of | % weight of | Tablet | |
|---|---|---|---|---|---|---|
| | % weight | Na ibuprofen | auxillary | auxillary | hardness | Disintegration |
| Example | of water^{a)} | hydrate | material(s) A^{b)} | material(s) B^{c)} | [N] | time [min] |
| 3 | 13.3% | 100% | — | — | 48 | 4.8 |
| 4 | 14.2% | 100% | — | — | 53 | 5.6 |
| 5 | 12.2% | 100% | — | — | 42 | 4.9 |
| 6 | 10.5% | 100% | — | — | 38 | 4.6 |
| 7 | 11.2% | 98.5% | — | 1.5% Mg stearate | 32 | 16.5 |
| 8 | 13.2% | 99.5% | — | 0.5% Mg stearate | 38 | 12.4 |
| 9 | 13.2% | 89.5% | — | 10.5% NaHCO₃ | 58 | 5.8 |
| 10 | 13.2% | 91.2% | 8.9% Povidone K25 | — | 64 | 7.8 |
| 11 | 14.1% | 91.2% | 8.9% Povidone K25 | — | 73 | 8.4 |
| 12 | 13.2% | 83.7% | 16.3% Povidone K25 | — | 89 | 9.6 |
| 13 | 13.3% | 83.4% | 7.5% Povidone K25 | 9.1% NaHCO₃ | 68 | 4.0 |
| 14 | 13.3% | 83.4% | 7.5% Povidone K25 | 9.1% KHCO₃ | 64 | 3.8 |
| 15 | 13.3% | 77.4% | 7.5% Povidone K25 | 15.1% Na₃ citrate | 69 | 5.8 |
| 16 | 13.3% | 77.4% | 17.5% Povidone K25 | 5.1% Na₃PO₄ | 72 | 6.2 |
| 17 | 13.3% | 77.4% | 7.5% Povidone K25 | 15.1% Na₂CO₃ | 66 | 6.8 |
| 18 | 13.3% | 77.4% | 7.5% Povidone K25 | 7.5% NaHCO₃, | 64 | 5.8 |
| | | | | 7.6% KHCO₃ | | |
| 19 | 13.3% | 76.6% | 7.5% Povidone K25 | 14.9% NaHCO₃ | 68 | 5.4 |
| | | | 1.0% Na | | | |
| | | | dodecylsulfate | | | |
| 20 | 12.7% | 77.3% | 7.6% Povidone K25 | 15.1% NaHCO₃ | 75 | 5.8 |
| 21 | 13.3% | 83.7% | 16.3% maltodextrin | — | 62 | 5.4 |
| 22 | 13.3% | 82.4% | 8.0% maltodextrin | 9.6% NaHCO₃ | 66 | 5.9 |
| 23 | 13.3% | 77.3% | 7.6% maltodextrin | 15.1% NaHCO₃ | 78 | 5.4 |
| 24 | 13.3% | 67.0% | 4.0% Povidone K25 | 10.5% microcryst. | 53 | 8.8 |
| | | | | cellulose, | | |
| | | | | 15.8% NaHCO₃, | | |
| | | | | 2.7% talc | | |
| 25 | 13.3% | 64.0% | 4.0% Povidone K25 | 10.5% microcryst. | 58 | 8.7 |
| | | | | cellulose, | | |
| | | | | 15.8% NaHCO₃, | | |
| | | | | 2.7% talc, | | |
| | | | | 3.0% Kollidon CL | | |
| 26 | 13.3% | 72.0% | 28.0% NaHCO₁ | — | 82 | 6.9 |
| 27 | 13.3% | 77.4% | 7.5% Povidone K25, | — | 69 | 5.1 |
| | | | 15.1% NaHCO₃ | | | |
| 28 | 12.7% | 82.3% | 8.0% Povidone K25^{d)} | 9.7% NaHCO₃ | 67 | 3.2 |
| 29 | 12.7% | 82.3% | 8.0% Povidone K25^{e)} | 9.7% NaHCO₃ | 83 | 3.4 |
| 30 | 12.7% | 82.3% | 8.0% Povidone K25^{f)} | 9.7% NaHCO₃ | 70 | 4.0 |
| 31 | 13.7% | 89.5% | 10.5% NaHCO₃ | — | 61 | 4.7 |
| 32 | 12.7% | 82.3% | 8.0% sorbitol | 9.7% NaHCO₃ | 71 | 4.0 |
| 33 | 12.7% | 89.5% | 10.5% sorbitol | — | 60 | 4.5 |
| 34 | 12.7% | 76.3% | 14.8% Povidone K25 | 8.9% NaHCO₃ | 89 | 5.2 |
| 35 | 12.7% | 76.3% | 14.8% sorbitol | 8.9% NaHCO₃ | 75 | 2.8 |
| 36 | 12.7% | 76.3% | 14.8% sorbitol | 8.9% Na₂CO₃ | 72 | 2.0 |
| 37 | 11.2% | 85.1% | 8.3% sorbitol | 6.6% NaHCO₃ | 68 | 4.3 |
| 38 | 6.0% | 85.1% | 8.3% sorbitol | 6.6% NaHCO₃ | 50 | 4.7 |
| 39 | 6.0% | 80.8% | 8.5% maltodextrin | 10.2% NaHCO₃, | 60 | 7.8 |
| | | | | 0.5% Mg stearat | | |
| 40 | 0.5% | 75.0% | 8.3% Povidone K25 | 16.7% NaHCO₃ | 43 | 4.2 |
| 41 | 12.7% | 82.4% | 8.0% maltodextrin^{g)} | 9.6% NaHCO₃ | 71 | 3.2 |
| 42 | 12.7% | 74.7% | 7.4% Povidone K25 | 14.9% NaHCO₃, | 89 | 9.5 |
| | | | | 3.0% SiO₂ | | |
| 43 | 12.7% | 74.7% | 7.4% Povidone K25 | 14.9% NaHCO₃, | 86 | 8.8 |
| | | | | 3.0% talc | | |
| 44 | 13.1% | 75.1% | 7.3% Povidone K25 | 8.8% NaHCO₃, | 72 | 4.4 |
| | | | | 8.8% NaCl | | |
| 45 | 13.3% | 75.1% | 7.3% Povidone K25 | 8.8% NaHCO₃, | 76 | 4.3 |
| | | | | 8.8% mannitol | | |
| 46 | 12.7% | 77.3% | 7.6% Povidone K25 | 14.8% NaHCO₃, | 64 | 9.2 |
| | | | | 0.3% dimethicone | | |
| 47 | 12.7% | 53.0% | 20.0% Povidone K25, | 22.0% NaHCO₃ | 115 | 7.2 |
| | | | 5.0% mannitol | | | |
| 48 | 12.7% | 53.0% | 20.0% Povidone K25, | — | 105 | 7.8 |
| | | | 5.0% mannitol, | | | |
| | | | 22.0% NaHCO₃ | | | |
| 49 | 13.2% | 70.0% | — | 15.0% NaCl, | 88 | 6.2 |
| | | | | 15.0% NaHCO₃ | | |
| 50 | 12.9% | 79.7% | 3.9% Povidone K25, | 12.5% NaHCO₃ | 65 | 4.2 |
| | | | 3.9% maltodextrin | | | |

^{a)}water content of the sodium ibuprofen hydrate, measured as loss on drying at 105° C.
^{b)}auxillary material(s) in the granulate
^{c)}tabletization auxillary material(s)
^{d)}granular size of the granulate in the range of 0.25 to 1.25 mm
^{e)}granular size of the granulate in the range of 0 to 0.25 mm
^{f)}granular size of the granulate in the range of 0 to 1.25 mm
^{g)}without compaction

To determine the crushing strength of the tablets, the necessary force to crush the tablets between the motorised jaws of a Schleuniger Hardness Tester was measured. The values reported in Table 1 are in each case the mean of 10 measurements.

The disintegration time of the tablets was measured by means of the disintegration method described in the European Pharmacopoeia, 4^{th} edition, Chapter 2.9.1, page 191, using water (pH about 7) as disintegration medium. The disintegration times listed in table 1 are in each case the mean of 6 measurements.

The formulation according to Example 40 proved to be extremely sticky on the tabletization tools and had a strong tendency to capping. A tendency to capping was also observed in the Examples 7 and 39 and furthermore sometimes also in the Examples 6, 37 and 38. In addition Examples 6, 38 and 39 gave formulations that stuck on the tabletization tools, and the formulation in Example 7 was sometimes sticky, although both effects were clearly less marked than in Example 40. The formulations according to Examples 42 and 43 were sticky (without a tendency to capping), which was also observed sometimes for those of the Examples 5, 24, 25 and 46. The formulations according to Examples 3 and 21 were only slightly sticky and showed no tendency to capping. The formulations according to Examples 9-20, 22, 23, 26-36, 41, 44, 45 and 47-50 showed good to very good tablet properties (hardness, disintegration time, friability, look of the tablet surface), in particular those of the Examples 12, 15-20, 22, 23, 29, 34, 47 and 48 resulting in practically perfect tablets. The surfaces of the tablets were perfectly smooth, nearly free of pores and very well suited for the film coating.

The influence of the water content showed itself particularly in Examples 3-6, in which the pure active ingredient was compressed. Good to acceptable tablets were obtained, if the water content was at least 11% by weight. If the water content sinks under this value, the tablets increasingly stick on the press tools, and the tablets have only a low crushing strength and show a tendency to capping. The sticking on the press tools can not be avoided through the addition of the highly effective anti-sticking agent magnesium stearate; rather through this addition the hardness of the tablets is drastically reduced and the disintegration time increases significantly over 10 minutes, as illustrated in Examples 7 and 8. Also the negative influence of an insufficient water content can only be compensated for in a limited manner by the addition of fillers and basic auxiliary materials, such as illustrated in Examples 38-40.

As Examples 24 and 25 show, the results of the tablet formulations, which additionally contain microcrystalline cellulose, talc and, if applicable, the disintegrant material Kollidon CL, are worse than comparable examples without these additions. The tablet hardness is not improved through these additions, and the disintegration time is about 9 minutes.

As the remaining examples verify, tablets with sufficient mechanical strength, disintegration times less than 10 minutes, mostly between about 2 and 7 minutes, and tablet hardness, depending on the quantity of auxiliary materials employed, of between about 50 and 120 N, are obtained by use of sufficient water content and by use of one or more fillers and/or basic auxiliary materials.

b) In an analogous manner to Example 1b, the tablets obtained in the Examples 4, 11, 13, 19-23, 30, 45, 47, 49 and 50 were provided with a film coat. The final weight of the film tablets was about 317-367 mg. Moreover, film coats were successfully produced, which contain as film formers carrageenan, polyvinyl alcohol and hydroxypropylmethylcellulose, as well as the usual plasticizers such as polyethylene glycol, triethyl citrate and triacetine.

The bioavailability of the film tablets obtained according to Examples 19 and 22 (in the following indicated as Example 19b and 22b) was tested on 15 subjects, Nurofen tablets (Boots) containing 200 mg ibuprofen being used as reference formulation. The subjects each received 2 film tablets, or dragees. The results of the bioavailability studies are compiled in Table 2.

TABLE 2

| | Example 19b | Example 22b | Nurofen |
|---|---|---|---|
| Cₘₐₓ (μg/ml) | 46.4 ± 8.8 | 47.6 ± 8.7 | 36.8 ± 9.4 |
| AUC_{0-∞} (ng × h/ml) | 135.6 ± 23.5 | 127.5 ± 25.5 | 130.7 ± 26.9 |
| tₘₐₓ (h) | 0.67 ± 0.4 | 0.62 ± 0.3 | 1.4 ± 1.1 |

As ibuprofen and the ibuprofen salts are absorbed in the entire intestinal tract, it is not surprising that all three preparations show almost the same bioavailability. On the other hand it is obvious from the Cₘₐₓ values that the formulations of this invention produce higher maximal blood levels. Particularly noticeable is the big difference in the times observed to achieve the maximal blood level, tₘₐₓ. The formulations of this invention are clearly superior to the reference sample. A significantly faster increase in blood level occurs and the maximum is reached around 45 minutes earlier. For a pain relieving medicine this is of great importance. With an achievement of the maximal blood level that is too late, the patient can be tempted to take a further tablet, since the pain relief begins too late.

### EXAMPLE 51 (DISSOLUTION TEST)

The active ingredient release from the tablets obtained in Examples 3-50 and from film tablets was tested by means of the method described in the European Pharmacopoeia, 4^{th} edition, Chapter 2.9.3, page 194, (Paddle Equipment) in the following three media:
1000 ml of 0.1 M hydrochloric acid (artificial gastric juice, pH 1.2),
1000 ml McIlvain Buffer (pH 3.5), produced from 702 ml 0.1 M aqueous citric acid solution and 298 ml 0.2 M aqueous Na₂HPO₄ solution;
1000 ml USP Buffer (pH 7.2), produced from 50 ml 0.2 M aqueous KH₂PO₄ solution and 34.7 ml 0.2 M aqueous NaOH solution, and made up with water to 1000 ml

The dissolution profiles of some formulations are graphically presented in FIGS. 2-5 for illustration. FIG. 2 shows the dissolution profile, which was measured by the peddle method in 0.1 M hydrochloric acid at 50 rpm, of the non-coated tablets (tablet cores) according to Examples 13, 14, 21, 22 and 33 (in the following and in FIG. 2 referred to as Example 13a, 14a, 21a, 22a, and 33a respectively) and the film tablet according to Example 50 (in the following and in FIG. 2 indicated as Example 50b). FIGS. 3-5 show the dissolution profiles, which were measured by the paddle method in the aforementioned media, of the film tablets according to the Examples 19, 20 and 22 (in the following and in FIGS. 3-5 referred to as Examples 19b, 20b and 22b respectively) and for comparison the corresponding dissolution profiles of Dolormin (Woelm Pharma, Germany), a preparation available on the market, a film tablet containing 342 mg ibuprofen lysinate, and Nurofen (Boots, Great Britain), a dragee coated with sugar, containing 200 mg ibuprofen in the form of the acid; FIG. 3 shows the dissolution profiles in 0.1 M hydrochloric acid at 100 rpm, FIG. 4 the dissolution profile in McIlvain Buffer at 100 rpm and FIG. 5 the dissolution profile in USP Buffer at 50 rpm.

Ibuprofen is an organic acid with a strongly pH-dependant solubility. In the pH range of 1-5 the solubility is significantly under 0.1 g/l. Only after pH 6 does it greatly increase as a consequence of salt formation and it reaches a value of about 20 g/l at pH 7. If the in vitro release is measured at pH 7.2, it is not surprising that for the Nurofen tablet, which contains the ibuprofen in the form of the acid, a rapid active ingredient release is likewise observed. Even at pH 7.2, the active ingredient release from the film tablets of this invention is however rapider than the release from the ibuprofen lysinate film tablet Dolormin, and in particular than the release from the ibuprofen film tablet Nurofen. However this difference at pH 7.2 can not to explain why the maximum blood level was reached with the formulations of this invention about 45 minutes faster than that with Nurofen.

However, an explanation for the significantly rapider blood level increase achieved in accordance with this invention is offered by the dissolution behaviour at acidic pH values (FIGS. 2-4). Due to the poor solubility of the ibuprofen at pH values under 5 and the limited volume of the dissolution medium, the active ingredient in these experiments was not completely dissolved. For Dolormin and Nurofen a comparatively slow, gradual dissolution was observed, such as illustrated in FIGS. 3 and 4. In contrast, the formulations of this invention showed an significantly improved dissolution behaviour, the film tablets according to Examples 20b and 22b tending to formation of highly supersaturated solutions in particular at pH 1.2 (without the pH values of the dissolution media being changed thereby). The drop in the curves after about 10-20 minutes is a consequence of the gradual crystallisation of ibuprofen, whereby the supersaturation is gradually reduced. It is assured that the supersaturation phenomenon also plays an important role in the observed, excellent in vivo resorption and that the supersaturated solutions might have an even significantly greater stability than under in vitro conditions due to the complex compositions of gastric and intestinal juices.

1-34. (canceled)

35. A non-effervescent tablet for oral administration of sodium ibuprofen, comprising a tablet core consisting of from 50% to 100% by weight of sodium ibuprofen hydrate and from 0% to 50% by weight of auxiliary material component, based on the weight of the tablet core which core contains no lubricant and no disintegrant, with the sodium ibuprofen hydrate having a water content of about 8% to about 16% by weight of the hydrate.

36. (canceled)

37. The tablet of claim 35 wherein the tablet contains no lubricant and no disintegrant.

38. The tablet as claimed in claim 35, wherein the water content of the sodium ibuprofen hydrate is 11 to 16% by weight of the hydrate.

39. The tablet as claimed in claim 38, wherein the water content of the sodium ibuprofen hydrate is 12.5 to 15% by weight of the hydrate.

40. The tablet as claimed in claim 35, wherein the sodium ibuprofen hydrate is present in an amount of from 50 to 99.9% by weight, based on the weight of the tablet core.

41. The tablet as claimed in claim 35, wherein the sodium ibuprofen hydrate is present in an amount of at least 60% by weight, based on the weight of the tablet core.

42. The tablet as claimed in claim 41, wherein the sodium ibuprofen hydrate is present in an amount of from 60 to 93% by weight, based on the weight of the tablet core.

43. The tablet as claimed in claim 42, wherein the sodium ibuprofen hydrate is present in an amount of at least 70% by weight, based on the weight of the tablet core.

44. The tablet as claimed in claim 43, wherein the sodium ibuprofen hydrate is present in an amount of from 70 to 85% by weight, based on the weight of the tablet core.

45. The tablet as claimed in claim 35, wherein the auxiliary material component comprises one or more basic auxiliary materials.

46. The tablet as claimed in claim 45, wherein the auxiliary material component comprises one or more water soluble, basic auxiliary materials.

47. The tablet as claimed in claim 46, wherein the auxiliary material component comprises one or more basic auxiliary materials, selected from basic alkali metal salts, basic alkaline earth metal salts, basic ammonium salts and basic amino acids.

48. The tablet as claimed in claim 47, wherein the auxiliary material component comprises one or more basic auxiliary materials, selected from sodium hydrogen carbonate, potassium hydrogen carbonate, sodium carbonate, potassium carbonate, trisodium citrate and trisodium phosphate.

49. The tablet as claimed in claim 48, wherein the auxiliary material component comprises at least one basic auxiliary material, selected from sodium hydrogen carbonate and potassium hydrogen carbonate.

50. The tablet as claimed in claim 45, wherein the proportion of the basic auxiliary material is from 5 to 30% by weight, based on the weight of the tablet core.

51. The tablet as claimed in claim 50, wherein the proportion of the basic auxiliary material is 6 to 25% by weight, based on the weight of the tablet core.

52. The tablet as claimed in claim 35, wherein the auxiliary material component comprises one or more neutral to weakly acidic fillers that improve the compressibility.

53. The tablet as claimed in claim 35, wherein the auxiliary material component comprises one or more water soluble, neutral to weakly acidic fillers that improve the compressibility.

54. The tablet as claimed in claim 35, wherein the auxiliary material component comprises one or more fillers, selected from sugars, hexoses, hydrolysed or enzymatically split starches, cyclodextrins, non-crosslinked polyvinylpyrrolidone, neutral to weakly acidic alkali metal salts, neutral to weakly acidic alkaline earth metal salts, and neutral to weakly acidic ammonium salts.

55. The tablet as claimed in claim 54, wherein the auxiliary material component comprises one or more fillers, selected from hexoses, non-crosslinked polyvinylpyrrolidone, maltodextrin and sodium chloride.

56. The tablet as claimed in claim 55, wherein the auxiliary material component comprises non-crosslinked polyvinylpyrrolidone as filler.

57. The tablet as claimed in claim 52, wherein the proportion of the filler is from 1 to 25% by weight, based on the weight of the tablet core.

58. The tablet as claimed in claim 57, wherein the proportion of the filler is 3 to 20% by weight, based on the weight of the tablet core.

59. The tablet as claimed in claim 35, wherein the auxiliary material component comprises one or more basic auxiliary materials and one or more neutral to weakly acidic fillers that improve the compressibility.

60. The tablet as claimed in claim 59, wherein the auxiliary material component comprises at least one basic auxiliary material, selected from sodium hydrogen carbonate and potassium hydrogen carbonate, and non-crosslinked polyvinylpyrrolidone as filler.

61. The tablet as claimed in claim 60, wherein the auxiliary material component comprises, based on the weight of the tablet core, from 5 to 15% of basic auxiliary material, selected from sodium hydrogen carbonate and potassium hydrogen carbonate, and from 7 to 20% of non-crosslinked polyvinylpyrrolidone as filler.

62. The tablet as claimed in claim 35, wherein the auxiliary material component consists of basic auxiliary material.

63. The tablet as claimed in claim 35, wherein the tablet core consists of sodium ibuprofen hydrate, the sodium ibuprofen hydrate has a water content of 11 to 16% by weight, and the hardness of the tablet is at least 30 N.

64. The tablet as claimed in claim 63, wherein the sodium ibuprofen hydrate has a water content of 12.5 to 15% by weight.

65. The tablet as claimed in claim 63, wherein the hardness of the tablet is at least 40 N.

66. The tablet as claimed in claim 35, wherein the sodium ibuprofen hydrate is present in racemic form.

67. The tablet as claimed in claim 35, wherein the sodium ibuprofen hydrate is present in the form of sodium S (+)-ibuprofen hydrate.

68. The tablet as claimed in claim 35, wherein the tablet core is coated with a sugar or film coat.

69. The tablet as claimed in claim 68, wherein the tablet core is coated with a sugar or film coat in an amount of from 1 to 10% by weight, based on the weight of the tablet core.
